(19) United States
(12) Patent Application Publication
(10) Pub. No.: US 2021/0403554 A1
(43) Pub. Date: Dec. 30, 2021

(54) METHODS FOR REDUCTION OF PATHOLOGICAL FIBROSIS AND ASSOCIATED DISORDERS AND DISEASES

(71) Applicants: EVOTEC SE, Hamburg (DE); THE BRIGHAM AND WOMEN'S HOSPITAL, INC., Boston, MA (US)

(72) Inventors: Uwe Andag, Duderstadt (DE); Benjamin D. Humphreys, Clayton, MO (US)

(21) Appl. No.: 17/463,065

(22) Filed: Aug. 31, 2021

Related U.S. Application Data
(63) Continuation of application No. 15/756,814, filed on Mar. 1, 2018, now abandoned.
(60) Provisional application No. 62/214,397, filed on Sep. 4, 2015.

(51) Int. Cl. C07K 16/28; A61K 31/7105; A61K 31/711; A61K 31/713

(52) U.S. Cl. CPC C07K 16/28; A61K 31/7105; C07K 2317/34; A61K 31/713; A61K 31/711

(57) ABSTRACT

Agents and methods for treating, reducing or ameliorating pathological fibrosis and disease or disorders caused by or associated with excessive deposition of fibrous tissue, and methods of identifying suitable agents. Preferred agents modulate the level and/or activity of GPR124.

### BACKGROUND OF THE INVENTION

### 1. Field of the Invention

G-protein coupled receptor (“GPCR”) family members are characterized by an extended extracellular region with a variable number of protein domains coupled to a TM7 domain via a domain known as the GPCR-autoproteolysis inducing (“GAIN”) domain. G-protein coupled receptor 124 (“GPR124”) is a protein that is encoded by the GPR124 gene in humans. GPR124 is a member of the adhesion-GPCR family of receptors.

### 2. Brief Description of the Background Art

WO 2002/083874 A2 discloses a broad variety of markers, including tumor endothelial marker 3 (“TEM3”).

WO 2003/033652 A2 discloses TEM5a polypeptides and nucleic acid molecules encoding the same.

WO 2003/046127 A2 discloses TEM5 polypeptides and nucleic acid molecules encoding the same. The invention also provides selective binding agents, vectors, host cells and methods for producing TEM5 polypeptides. It further provides pharmaceutical compositions and methods for the diagnosis, treatment, amelioration, or prevention of diseases associates with TEM5 polypeptides.

Carson-Walter characterized selected TEMs (including TEM5) and also identified mouse counterparts of these. (Carson-Walter et al., Cancer Research, Vol. 61 (2001) 6649-55.)

Nagase predicted sequences of 100 cDNA clones of unknown human genes which have the potential to code for large proteins in vitro from two sets of size-fractionated human adult and fetal brain cDNA libraries. Among these genes was identified KIAA1531. (Nagase et al., DNA Research, Vol. 7 (2000) 143-50.)

Posokhova describes GPR124 as a WNT7-specific coactivator of b-catenin signaling in brain endothelium. The authors map areas within GPR124 (PDZ binding motif & leucine-rich domain of extracellular domain) that are required for WNT7/b-catenin signaling. (Posokhova et al., Cell Reports, Vol. 10, No. 2 (2015) 123-30.)

St. Croix identified transcripts corresponding to several tumor endothelial markers that displayed elevated expression during tumor angiogenesis using serial analysis of gene expression, including TEM5. (St. Croix et al., Science, 289 (2000) 1197-202.)

Vallon describes thrombin-induced shedding of tumour endothelial marker 5 and exposure of its RGD motif being regulated by cell-surface protein disulfide-isomerase. (Vallon et al., Biochem. J., (2012) 441, 937-944.)

ADGRA2 is an adhesion G protein-coupled receptor A2 identified from European shrew (Sorex araneus), Gene ID: 101547491.

### SUMMARY OF THE INVENTION

It is an object of the present invention to provide a method of reducing pathological fibrosis in a mammalian subject.

It is another object of the present invention to provide a method of treating, preventing or ameliorating a disorder or disease associated with or caused by excessive deposition of fibrous tissue in a mammalian subject.

It is yet another object of the present invention to provide a use of an agent that modulates level and/or activity of GPR124.

It is a further object of the present invention to provide an agent that modulates level and/or activity of GPR124.

It is a still further object of the present invention to provide a method of identifying therapeutic agents that inhibit the transition of cells to myofibroblasts.

It is another object of the present invention to provide a method of identifying therapeutic agents that reduce fibrosis.

It is an object of the present invention to provide a method of identifying therapeutic agents that treat, prevent or ameliorate a disorder or disease associated with or caused by excessive deposition of fibrous tissue.

It is yet another object of the present invention to provide a method of identifying therapeutic agents that inhibit the transition of cells to myofibroblasts and/or reduce fibrosis.

These objects and others are provided by the present invention, which relates to novel disease associations of GPR124 polypeptides and polynucleotides. The present invention also relates to methods of screening for therapeutic agents for the treatment of pathological fibrosis as well as disorders and diseases associated with or caused by an excessive deposition of fibrous tissue. The present invention further relates to agents for the treatment of pathological fibrosis as well as disorders and diseases associated with or caused by an excessive deposition of fibrous tissue.

The present invention is intended to encompass suitable agents, methods of identifying and uses of such agents. Methods of identifying suitable agents include determining whether test agents reduce the expression level of one or more myofibroblast markers and/or the production or deposition levels of extracellular matrix component as compared to reference. Alpha-smooth muscle actin may be utilized as the myofibroblast marker. Collagen, such as collagen type 1, may be utilized as the extracellular matrix component. To optimize efficiency and detection of therapeutic benefit, it is preferred that test cells overexpress GPR124. Similarly, test agents may be evaluated by culturing cells in the presence of an inducer of myofibroblast transition, such as transforming growth factor-beta or a ligand of the GPR124 receptor. Cells may be contacted with the test agent and the myofibroblast transition inducer sequentially; if so the cells are preferably contacted with the test agent prior to being contacted with the myofibroblast transition inducer.

### BRIEF DESCRIPTION OF THE DRAWINGS

FIG. 1 illustrates the upregulation of GPR124 in a mouse fibrosis model;

FIG. 2 illustrates the upregulation of GPR124 in a mouse diabetic nephropathy model;

FIG. 3 illustrates the upregulation of GPR124 mRNA expression in NRK49F pericytes following treatment with transforming growth factor-beta (“TGF-beta”);

FIGS. 4(A) and 4(B) illustrate GPR124 overexpression induces NRK49F pericyte-to-myofibroblast transition (“PMT”);

FIG. 5(A) shows GPR124 expression in adult sham control kidney whereas FIG. 5(B) shows GPR124 expression in adult kidney at day 10 after Unilateral Ureteral Obstruction (UUO), a kidney fibrosis model.

FIG. 6 illustrates a comparison of the amino acid sequences HsGPR124 and MmGPR124.

### DETAILED DESCRIPTION OF THE INVENTION

GPR124 is also known in the art as KIAA1531, adhesion G-protein coupled receptor 2 (“ADGRA2”) and tumor endothelial marker 5 (“TEM5”). GPR124 has been shown to interact with Disks large homolog 1 (“DLG1”, also known as synapse-associated protein 97 or “SAP97”), a protein that is encoded by the SAP97 gene in humans. SAP97 is a mammalian membrane-associated guanylate kinase (“MAGUK”)-family member protein that is similar to the Drosophila protein Dlg1. SAP97 is expressed throughout the body in epithelial cells and is involved in the brain in the trafficking of ionotropic receptors from the endoplasmic reticulum to the plasma membrane. SAP97 may also be involved in the trafficking the α-amino-3-hydroxy-5-methyl-4-isoxazolepropionic acid receptor (“AMPAR”, also known as the quisqualate receptor). AMPAR is a non-NMDA-type ionotropic transmembrane glutamate receptor that mediates fast synaptic transmission in the central nervous system.

GPR124 is a member of a class of Leu rich repeat (“LRR”) GPCRs, which have a large N-terminal extracellular domain. The LRR of GPR124 has relatively high homology with LRIG1 and SLIT1/2. Consistent with the pattern of expression on endothelial cells and pericytes, knockout animal studies have underwritten a role for GPR124 in CNS vasculogenesis. GPR124 was originally identified as a gene overexpressed in tumor vessels of human colorectal carcinoma and cell based studies have suggested that GPR124 is important in endothelial cell migration by regulating VEGF expression and promoting vessel leakage.

Global or endothelial-specific deletion of GPR124 in mice results in embryonic lethality associated with abnormal angiogenesis of the forebrain and spinal cord. Expression of GPR124 was found to be required for invasion and migration of blood vessels into neuroepithelium, establishment of blood brain barrier properties, and expansion of the cerebral cortex. Therefore, GPR124 is understood to regulate neurovasculature development.

As utilized herein, GPR124 includes polypeptides encoded by the nucleotide sequences SEQ ID NO:1 and SEQ ID NO:2, as well as polypeptides homologous thereto. The terms “polypeptide,” “peptide” and “protein” are used interchangeably herein to refer to a polymer of amino acid residues. The terms apply to amino acid sequences or polymers in which one or more amino acid residue is an artificial chemical mimetic of a corresponding naturally occurring amino acid, as well as to naturally occurring amino acid polymers, those containing modified residues, and non-naturally occurring amino acid polymer.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar naturally occurring and non-naturally occurring amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Homologous,” in relation to two or more peptides, refers to two or more sequences or subsequences that have a specified percentage of amino acid residues that are the same over a specified region, when compared and aligned for maximum correspondence over a comparison window or designated region as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids in length, or more preferably over a region that is 50-100 amino acids in length. Over the full length of the GPR124 sequence the suitable percentage of amino acid residues that are the same may be at least about 50% identity, preferably 55%, 60%, 65%, 70%, 75%, 80%, 85%, 90%, 91%, 92%, 93%, 94%, 95%, 96%, 97%, 98%, 99%, or higher. For fragments of the GPR124 protein the suitable percentage of amino acid residues that are the same may be at least 60% identity, preferably 65%, 70%, 75%, 80%, 85%, 90%, 91%, 92%, 93%, 94%, 95%, 96%, 97%, 98%, 99%, or higher.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., Vol. 2 (1981) 482, by the homology alignment algorithm of Needleman & Wunsch, J. Mol. Biol., Vol. 48 (1970) 443, by the search for similarity method of Pearson & Lipman, Proc. Nat'l. Acad. Sci. USA, Vol. 85 (1988) 2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res., Vol. 25 (1977) 3389-402 and Altschul et al., J. Mol. Biol., Vol. 215 (1990) 403-10. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, Vol. 89 (1989) 10915) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, Vol. 90 (1993) 5873-87). One measure of similarity provided by the BLAST algorithm is the smallest sum probability (P(N)), which provides an indication of the probability by which a match between two nucleotide or amino acid sequences would occur by chance. For example, a peptide is considered similar to a reference sequence if the smallest sum probability in a comparison of the test peptide to the reference peptide is less than about 0.2, more preferably less than about 0.01, and most preferably less than about 0.001. Log values may be large negative numbers, e.g., 5, 10, 20, 30, 40, 40, 70, 90, 110, 150, 170, etc.

This invention concerns G-protein coupled receptor GPR124 and homologous peptides, and their use as a therapeutic target in pathological fibrosis as well as in disorders and diseases associated with or caused by an excessive deposition of fibrous tissue. The inventors have discovered that GPR124 is expressed in kidney pericytes, and that expression rises in pericytes during fibrotic kidney disease. The inventors' data shows that GPR124 expression in pericytes both sensitizes these cells to the pro-fibrotic effects of TGF-beta and independently drives myofibroblast transition. Since myofibroblasts are the critical cell type in fibrotic organ disease, these observations strongly suggest that antagonists of GPR124 would be antifibrotic, not only in kidney but also in other organs such as liver, lung, heart and skin.

In one aspect, the present invention is directed to a method for reducing pathological fibrosis in a mammalian subject in need thereof, comprising modulating the level and/or activity of G-protein coupled receptor 124 (GPR124) in said subject.

In a further aspect, the present invention is directed to a method for treating, preventing or ameliorating a disorder or disease associated with or caused by excessive deposition of fibrous tissue in a mammalian subject in need thereof, comprising modulating the level and/or activity of G-protein coupled receptor 124 (GPR124) in said subject.

In a further aspect, the present invention is directed to the use of an agent that modulates level and/or activity of G-protein coupled receptor 124 (GPR124) for reducing pathological fibrosis in a mammalian subject in need thereof.

In still a further aspect, the present invention is directed to the use of an agent that modulates level and/or activity of G-protein coupled receptor 124 (GPR124) for treating, preventing or ameliorating a disorder or disease associated with or caused by excessive deposition of fibrous tissue in a mammalian subject in need thereof.

In another aspect, the present invention is directed to the use of an agent that modulates level and/or activity of G-protein coupled receptor (GPR124) for the manufacture of a medicament for the reduction of pathological fibrosis in a mammalian subject in need thereof.

In still another aspect, the present invention is directed to the use of an agent that modulates level and/or activity of G-protein coupled receptor (GPR124) for the manufacture of a medicament for the treatment, prevention or amelioration of a disorder or disease associated with or caused by excessive deposition of fibrous tissue in a mammalian subject in need thereof.

In a further aspect, the present invention is directed to an agent that modulates level and/or activity of G-protein coupled receptor (GPR124) for use in a method for the reduction of pathological fibrosis in a mammalian subject in need thereof.

In yet a further aspect, the present invention is directed to an agent that modulates level and/or activity of G-protein coupled receptor (GPR124) for use in a method for the treatment, prevention or amelioration of a disorder or disease associated with or caused by excessive deposition of fibrous tissue in a mammalian subject in need thereof.

In these methods, uses and agents, the level and/or activity of GPR124 is preferably reduced, in particular employing one or more GPR124 antagonists.

Thus, the present invention is directed to methods for reducing pathological fibrosis and treating diseases or disorders associated with or caused by excessive deposition of fibrous tissue in mammals using materials that antagonizes GPR124 proteins. Such pathological fibrosis or disorder or disease may impair architecture and/or function of organs, such as kidney, liver, lung, heart or skin. Further organs which may be impaired include pancreas, vascular vessels, bone marrow and the like. Thus, for example, disorders or diseases (or pathological fibrosis associated therewith) to be treated according to the present invention are selected from the group consisting of diabetic nephropathy, focal segmental glomerulosclerosis (FSGS), minimal change disease (MCD), chronic kidney disease (CKD), end stage renal disease (ESRD), liver cirrhosis, pre-stage(s) of liver cirrhosis, idiopathic pulmonary fibrosis, pulmonary cystic fibrosis, myocardial fibrosis and scleroderma. In therapeutic use, GPR124 antagonists generally will be in the form of a pharmaceutical composition containing the antagonist and a pharmaceutically acceptable carrier. Pharmaceutically acceptable carriers are well known in the art and include aqueous solutions such as physiologically buffered saline or other buffers or solvents or vehicles such as glycols, glycerol, and/or oils such as olive oil or injectable organic esters. The selection of a pharmaceutically acceptable carrier will depend, in part, on the chemical nature of the GPR124 antagonist, for example, whether the GPR124 antagonist is an antibody, a peptide or a nonpeptide.

A pharmaceutically acceptable carrier may include physiologically acceptable compounds that act, for example, to stabilize the GPR124 antagonist or increase its absorption, or other excipients as desired. Physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the GPR124 antagonist and on its particular physio-chemical characteristics.

Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, maltose, sucrose or dextrins, chelating agents such as EDTA, glutathione and other stabilizers and excipients. Neutral buffered saline or saline mixed with nonspecific serum albumin are exemplary appropriate diluents.

The pharmaceutical compositions of the present invention may be prepared for administration by a variety of different routes. In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal, or intraurethral injection or infusion. A pharmaceutical composition (e.g., for oral administration or delivery by injection) may be in the form of a solid or a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers such as acetates, citrates or phosphates and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The methods of the present invention include application of GPR124 antagonists in cocktails including other medicaments, for example, blood pressure reducing agents, blood glucose reducing agents, blood lipid/cholesterol reducing agents, anti-coagulants, antibiotics, fungicides, and anti-inflammatory agents. Alternatively, the methods may comprise sequential dosing of an afflicted individual with a GPR124 antagonist and one or more additional medicaments to optimize a treatment regime. In such optimized regimes, the medicaments may be applied in any sequence and in any combination.

The GPR124 antagonists of the present invention may also be included in slow release formulations for prolonged treatment following a single dose. In one embodiment, the formulation is prepared in the form of microspheres. The microspheres may be prepared as a homogenous matrix of a GPR124 antagonist with a biodegradable controlled release material, with optional additional medicaments as the treatment requires. The microspheres are preferably prepared in sizes suitable for infiltration and/or injection, and injected systemically, or directly at the site of treatment.

The formulations of the invention are also suitable for administration in all body spaces/cavities, including but not limited to pleura, peritoneum, cranium, mediastinum, pericardium, bursae or bursal, epidural, intrathecal, intraocular, intra-articular, intra-discal, intra-medullary, perispinal, etc.

Some slow release embodiments include polymeric substances that are biodegradable and/or dissolve slowly. Such polymeric substances include polyvinylpyrrolidone, low- and medium-molecular-weight hydroxypropyl cellulose and hydroxypropyl methylcellulose, cross-linked sodium carboxymethylcellulose, carboxymethyl starch, potassium methacrylatedivinylbenzene copolymer, polyvinyl alcohols, starches, starch derivatives, microcrystalline cellulose, ethylcellulose, methylcellulose, and cellulose derivatives, β-cyclodextrin, poly(methyl vinyl ethers/maleic anhydride), glucans, scierozlucans, mannans, xanthans, alzinic acid and derivatives thereof, dextrin derivatives, glyceryl monostearate, semisynthetic glycerides, glyceryl palmitostearate, glyceryl behenate, polyvinylpyrrolidone, gelatine, agnesium stearate, stearic acid, sodium stearate, talc, sodium benzoate, boric acid, and colloidal silica.

Slow release agents of the invention may also include adjuvants such as starch, pregelled starch, calcium phosphate mannitol, lactose, saccharose, glucose, sorbitol, microcrystalline cellulose, gelatin, polyvinylpyrrolidone. methylcellulose, starch solution, ethylcellulose, arabic gum, tragacanth gum, magnesium stearate, stearic acid, colloidal silica, glyceryl monostearate, hydrogenated castor oil, waxes, and mono-, bi-, and trisubstituted glycerides. Slow release agents may also be prepared as generally described in WO94/06416 to Jagotec AG.

No treatments exist today that directly target kidney fibrosis, and the care of patients is directed at managing the complications of the disease, such as hypertension, disordered mineral metabolism and volume management. The present invention identifies GPR124 as a critical mediator of the fibrotic pericytes through its upregulation in kidney pericytes undergoing PMT and in myofibroblasts. Targeting this pathway would treat the cause of kidney fibrosis rather than the symptoms of disease, and therefore has the potential to slow or even reverse the course of disease. GPR124 has not been described in fibrosis, in particular fibrosis of the kidney, and it is a novel therapeutic target. Another novel aspect of this invention is the finding that GPR124 is upregulated in pericytes undergoing PMT and in myofibroblasts, the two most important cell types in the genesis of fibrosis.

The amount of GPR124 antagonist administered to an individual will depend, in part, on the disease to be treated and/or extent of injury. Methods for determining an effective amount of an agent to administer for a therapeutic procedure are well known in the art and include phase I, phase II and phase III clinical trials. Generally, an agent antagonist is administered in a dose of about 0.01 to 200 mg/kg body weight when administered systemically, and at a concentration of approximately 0.01-100 μM when administered directly to a wound site. The total amount of GPR124 antagonist can be administered to a subject as a single dose, for example either as a bolus or by infusion over a relatively short period of time, or can be administered using a fractionated treatment protocol, in which the multiple doses are administered over a more prolonged period of time. One skilled in the art would know that the concentration of a particular GPR124 antagonist required to provide an effective amount to a region or regions of injury depends on many factors including the age and general health of the subject as well as the route of administration, the number of treatments to be administered, and the nature of the GPR124 antagonist, including whether the GPR124 antagonist is an antibody, a peptide, or a nonpeptide molecule. In view of these factors, the skilled artisan would adjust the particular dose so as to obtain an effective amount for efficacious therapeutic purposes. Those of ordinary skill in this art are able to determine the appropriate “therapeutically effective amount” for administering such antagonists, as well as methods and schedules for such administration.

As disclosed herein, non peptides and proteins that antagonize specific binding of GPR124 to its natural ligand may serve as therapeutic agents of the present invention. Suitable proteins include antibodies, muteins and nucleic acid aptamers.

The phrase “specifically (or selectively) binds” or when referring to an antibody interaction, “specifically (or selectively) immunoreactive with,” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

Specific binding to an antibody under such conditions requires an antibody that is selected for its specificity for a particular protein For example, antibodies raised against a particular protein, polymorphic variants, alleles, orthologs, and conservatively modified variants, or splice variants, or portions thereof, can be selected to obtain only those antibodies that are specifically immunoreactive with GPR124 and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods for determining whether two molecules specifically interact are disclosed herein, and methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W.H. Freeman and Co. 1976)).

Furthermore, suitable antagonists can interfere with the specific binding of a receptor and its ligand by various mechanisms, including, for example, by binding to the ligand binding site, thereby interfering with ligand binding; by binding to a site other than the ligand binding site of the receptor, but sterically interfering with ligand binding to the receptor; by binding the receptor and causing a conformational or other change in the receptor, which interferes with binding of the ligand; or by other mechanisms. For purposes of the methods disclosed herein, an understanding of the mechanism by which the interference occurs is not required and no mechanism of action is proposed. A GPR124 antagonist such as an anti-GPR124 antibody, or antigen binding fragment thereof, is characterized by having specific binding activity (Kₐ) of at least about 10⁵M⁻¹, 10⁶M⁻¹ or greater, preferably 10⁷M⁻¹ or greater, more preferably 10⁸M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci., Vol. 51 (1949) 660-72).

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)2, Fab, Fv and rIgG). See also, Pierce Catalog and Handbook, 1994-1995 (Pierce Chemical Co., Rockford, Ill.). See also, e.g., Kuby, J., Immunology, 3rd Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science, Vol. 246 (1989) 1275-81. These and other methods of making, for example, chimeric, humanized, CDR-grafted, single chain, and bifunctional antibodies are well known to those skilled in the art (Winter and Harris, Immunol. Today, Vol. 14 (1993) 243-46; Ward et al., Nature, Vol. 341 (1989) 544-46; Harlow and Lane, supra, 1988; Hilyard et al., Protein Engineering: A practical approach (IRL Press 1992); Borrabeck, Antibody Engineering, 2d ed. (Oxford University Press 1995).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al., J. Immunol, Vol. 148 (1992) 1547; Pack and Pluckthun, Biochemistry, Vol. 31 (1992) 1579; Hollinger et al., supra; Gruber et al., J Immunol., Vol. 152, No. 11 (1994) 5368; Zhu et al., Protein Sci., Vol. 6 (1997) 781; Hu et al., Cancer Res., Vol. 56 (1996) 3055; Adams et al., Cancer Res., Vol. 53 (1993) 4026; and McCartney, et al., Protein Eng., Vol. 8 (1995) 301.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature, Vol. 321 (1986) 522-25; Riechmann et al., Nature, Vol. 332 (1988) 323-29; and Presta, Curr. Op. Struct. Biol., Vol. 2 (1992) 593-96). Humanization can be essentially performed following the method of Winter and co-workers (Jones et al., Nature, Vol. 321 (1986) 522-25; Riechmann et al., supra; Verhoeyen et al., Science, Vol. 239 (1988) 1534-36), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

These GPR124 recognizing antibodies may be made readily by those of ordinary skill in this art by conventional techniques. Preferably, these antibodies will be FAB fragments or monoclonal antibodies, and more preferably, the FAB fragments or monoclonal antibodies will be humanized.

Methods for producing both monoclonal and polyclonal antibodies from identified proteins or peptides are well known in the art. In order to prepare recombinant chimeric and humanized antibodies that may function as GPR124 antagonists of the present invention, the nucleic acid encoding non-human antibodies must first be isolated. This is typically done by immunizing an animal, for example a mouse, with prepared GPR124 or an antigenic peptide derived therefrom. Typically mice are immunized twice intraperitoneally with approximately 50 micrograms of the target protein per mouse. Sera from immunized mice can be tested for antibody activity by immunohistology or immunocytology on any host system expressing such polypeptide and by ELISA with the expressed polypeptide. For immunohistology, active antibodies of the present invention can be identified using a biotinconjugated anti-mouse immunoglobulin followed by avidin-peroxidase and a chromogenic peroxidase substrate. Preparations of such reagents are commercially available; for example, from Zymad Corp., San Francisco, Calif. Mice whose sera contain detectable active antibodies according to the invention can be sacrificed three days later and their spleens removed for fusion and hybridoma production. Positive supernatants of such hybridomas can be identified using the assays common to those of skill in the art, for example, Western blot analysis.

The nucleic acids encoding the desired antibody chains can then be isolated by, for example, using hybridoma mRNA or splenic mRNA as a template for PCR amplification of the heavy and light chain genes (Huse, et al., Science, Vol. 246 (1989) 1276). Nucleic acids for producing both antibodies and intrabodies can be derived from murine monoclonal hybridomas using this technique (Richardson J. H., et al., Proc Natl Acad Sci USA, Vol. 92 (1995) 3137-41; Biocca S., et al., Biochem and Biophys. Res. Comm., Vol. 197 (1993) 422-27; and Mhashilkar, A. M., et al., EMBO J., Vol. 14 (1995)1542-51). These hybridomas provide a reliable source of well-characterized reagents for the construction of antibodies and are particularly useful once their epitope reactivity and affinity has been characterized. Isolation of nucleic acids from isolated cells is discussed further in Clackson, T., et al., Nature, Vol. 352 (1991) 624-28 (spleen) and Portolano, S., et al., supra; Barbas, C. F., et al., supra; Marks, J. D., et al., supra; Barbas, C. F., et al., Proc Natl Acad Sci USA, Vol. 88 (1991) 7978-82 (human peripheral blood lymphocytes). Humanized antibodies optimally include at least a portion of an immunoglobulin constant region (Fc), typically that of a human immunoglobulin (Jones et al., Nature, Vol. 321 (1986) 522-25; Riechmann et al., Nature, Vol. 332 (1988) 323-29; and Presta, Curr. Op. Struct. Biol., Vol. 2 (1992) 593-96).

A number of methods have been described to produce recombinant antibodies, both chimeric and humanized. Controlled rearrangement of antibody domains joined through protein disulfide bonds to form chimeric antibodies may be utilized (Konieczny et al., Haematologia, 14(1):95-99, 1981). Recombinant DNA technology can also be used to construct gene fusions between DNA sequences encoding mouse antibody variable light and heavy chain domains and human antibody light and heavy chain constant domains (Morrison et al., Proc. Natl. Acad. Sci. USA, Vol. 81, No. 21 (1984) 6851-55).

DNA sequences encoding the antigen binding portions or complementarity determining regions (CDR's) of murine monoclonal antibodies may be grafted by molecular means into the DNA sequences encoding the frameworks of human antibody heavy and light chains (Jones et al., Nature, Vol. 321, No. 6069 (1986) 522-25; Riechmann et al., Nature, Vol. 332, No. 6162 (1988) 323-27). The expressed recombinant products are called “reshaped” or humanized antibodies, and comprise the framework of a human antibody light or heavy chain and the antigen recognition portions, CDR's, of a murine monoclonal antibody.

Other methods for producing humanized antibodies are described in U.S. Pat. Nos. 4,816,567; 4,935,496; 5,502,167; 5,530,101; 5,558,864; 5,565,332; 5,585,089; 5,639,641; 5,693,493; 5,693,761; 5,693,762; 5,698,417; 5,705,154; 5,733,743; 5,750,078 and 5,770,403, each incorporated herein by reference in their entirety.

Techniques described for the production of single chain antibodies (U.S. Pat. No. 4,946,778, which is incorporated by reference) can be adapted to produce single chain humanized antibodies to GPR124.

### 2. PURIFICATION OF RECOMBINANT ANTIBODY

Affinity purification of an antibody pool or sera provides a practitioner with a more uniform reagent. Methods for enriching antibody granulation inhibitors using antibody affinity matrices to form an affinity column are well known in the art and available commercially (AntibodyShop, c/o Statens Serum Institut, Artillerivej 5, Bldg. P2, DK-2300 Copenhagen S). Briefly, an antibody affinity matrix is attached to an affinity support (see e.g.; CNBR Sepharose (R), Pharmacia Biotech). A mixture comprising antibodies is then passed over the affinity matrix, to which the antibodies bind. Bound antibodies are released by techniques common to those familiar with the art, yielding a concentrated antibody pool. The enriched antibody pool can then be used for further immunological studies, some of which are described herein by way of example.

Another approach uses recombinant bacteriophage to produce large libraries. Using the “phage method” (Scott and Smith, Science, Vol. 249 (1990) 386-90; Cwirla, et al, Proc. Natl. Acad. Sci., Vol. 87 (1990) 6378-82; Devlin et al., Science, Vol. 49 (1990) 404-6), very large libraries can be constructed (106-108 chemical entities). A second approach uses primarily chemical methods, of which the Geysen method (Geysen et al., Molecular Immunology, Vol. 23 (1986) 709-15; Geysen et al. J. Immunologic Method, Vol. 102 (1987) 259-74; and the method of Fodor et al. (Science, Vol. 251 (1991) 767-73) are examples. Furka et al. (14th International Congress of Biochemistry, Vol. 5 (1988) Abstract FR:013; Furka, Int. J. Peptide Protein Res., Vol. 37 (1991) 487-93), Houghton (U.S. Pat. No. 4,631,211) and Rutter et al. (U.S. Pat. No. 5,010,175) describe methods to produce a mixture of peptides that can be tested as agonists or antagonists.

### 3. IDENTIFICATION OF GPR124 ANTAGONISTS

In one aspect, the present invention is directed to a method of identifying a therapeutic agent that is capable of inhibiting the transition of cells to myofibroblasts, comprising:
contacting mammalian cells with a test agent;
determining the effect of said test agent on level and/or activity of G-protein coupled receptor 124 (GPR124) in said cells; and
selecting a test agent that modulates the level and/or activity of GPR124 for inhibiting the transition of cells to myofibroblasts.
In a further aspect, the present invention is directed to a method of identifying a therapeutic agent for reducing fibrosis comprising:
contacting mammalian cells with a test agent;
determining the effect of said test agent on level and/or activity of G-protein coupled receptor 124 (GPR124) in said cells; and
selecting a test agent that modulates the level and/or activity of GPR124 for reducing fibrosis.

In still a further aspect, the present invention is directed to a method of identifying a therapeutic agent for treatment, prevention or amelioration of a disorder or disease associated with or caused by excessive deposition of fibrous tissue comprising:
contacting mammalian cells with a test agent;
determining the effect of said test agent on level and/or activity of G-protein coupled receptor 124 (GPR124) in said cells; and
selecting a test agent that modulates the level and/or activity of GPR124 for treating, preventing or ameliorating said disorder or disease.

In these methods, the selected test agent preferably decreases level and/or activity of GPR124. Selected test agents may serve as therapeutic agents according to the present invention.

In another aspect, the present invention is directed to a method of identifying a therapeutic agent for inhibiting the transition of cells to myofibroblasts and/or reducing fibrosis, comprising:

contacting mammalian cells expressing G-protein coupled receptor 124 (GPR124) with a test agent and with an inducer of myofibroblast transition under conditions allowing the cells to differentiate into myofibroblasts;

determining the expression level of one or more myofibroblast markers in the induced cells treated with the test agent;

comparing the expression level of the one or more myofibroblast markers in the induced cells treated with the test agent to a reference expression level; and

selecting a test agent that varies the expression level of said one or more myofibroblast markers in the induced cells as compared to the reference expression level for inhibiting the transition of cells to myofibroblasts and/or reducing fibrosis.

In still another aspect, the present invention is directed to a method of identifying a therapeutic agent for treatment, prevention or amelioration of a disorder or disease associated with or caused by excessive deposition of fibrous tissue, comprising:

contacting mammalian cells expressing G-protein coupled receptor 124 (GPR124) with a test agent and with an inducer of myofibroblast transition under conditions allowing the cells to differentiate into myofibroblasts;

determining the expression level of one or more myofibroblast markers in the induced cells treated with the test agent;

comparing the expression level of the one or more myofibroblast markers in the induced cells treated with the test agent to a reference expression level; and

selecting a test agent that varies the expression level of said one or more myofibroblast markers in the induced cells as compared to the reference expression level for treating, preventing or ameliorating said disorder or disease.

In a further aspect, the present invention is directed to a method of identifying a therapeutic agent for inhibiting transition of cells to myofibroblasts and/or reducing fibrosis, comprising:

contacting mammalian cells expressing G-protein coupled receptor 124 (GPR124) with a test agent and with an inducer of myofibroblast transition under conditions allowing the cells to differentiate into myofibroblasts;

determining the extent of production and/or extracellular deposition of one or more extracellular matrix (ECM) components in the induced cells treated with the test agent;

comparing the extent of production and/or extracellular deposition of said ECM components in the induced cells treated with the test agent to a reference level of ECM component production or deposition; and selecting a test agent that varies the extent of production and/or extracellular deposition of said ECM components in the induced cells as compared to a reference level of ECM component production or deposition for inhibiting transition of cells to myofibroblasts and/or reducing fibrosis.

In still a further aspect, the present invention is directed to a method of identifying a therapeutic agent for treatment, prevention or amelioration of a disorder or disease associated with or caused by excessive deposition of fibrous tissue, comprising:

contacting mammalian cells expressing G-protein coupled receptor 124 (GPR124) with a test agent and with an inducer of myofibroblast transition under conditions allowing the cells to differentiate into myofibroblasts;

determining the extent of production and/or extracellular deposition of one or more extracellular matrix (ECM) components in the induced cells treated with the test agent;

comparing the extent of production and/or extracellular deposition of the one or more ECM components in the induced cells treated with the test agent to a reference level of ECM component production and/or deposition; and selecting a test agent that varies the extent of production and/or extracellular deposition of said ECM components in the induced cells as compared to a reference level of ECM component production or deposition for treating, preventing or ameliorating said disorder or disease.

As disclosed herein, selected test agents may serve therapeutic agents according to the present invention.

In one embodiment of the herein disclosed methods, the reference expression level is the expression level of one or more myofibroblast markers in induced cells not treated with the test agent and a test agent is selected that decreases the expression level of one or more myofibroblast markers as compared to the reference expression level. The myofibroblast marker may e.g. be alpha-SMA.

In another embodiment of the herein disclosed methods, the reference level of ECM component production and/or deposition is the level of ECM component production and/or deposition in induced cells not treated with the test agent and a test agent is selected that decreases the extent of production and/or extracellular deposition of one or more ECM components as compared to the reference level of ECM component production and/or deposition. For example, the extracellular matrix component may be collagen.

In a further aspect, the present invention is directed to a method of identifying a therapeutic agent for inhibiting transition of cells to myofibroblasts and/or reducing fibrosis, comprising:

contacting a test agent with G-protein coupled receptor (GPR124) or a fragment thereof;

detecting binding of said test agent to GPR124 or the fragment thereof; and

selecting a test agent that binds to GPR124 or the fragment thereof for inhibiting transition of cells to myofibroblasts and/or reducing fibrosis.

In still a further aspect, the present invention is directed to a method of identifying a therapeutic agent for treatment, prevention or amelioration of a disorder or disease associated with or caused by excessive deposition of fibrous tissue, comprising:

contacting a test agent with G-protein coupled receptor (GPR124) or a fragment thereof;

detecting binding of said test agent to GPR124 or the fragment thereof; and

selecting a test agent that binds to GPR124 or the fragment thereof for treating, preventing or ameliorating said disorder or disease.

With respect to the aforementioned binding assays, the method may further comprise contacting GPR124 or the fragment thereof with a ligand and evaluating whether the test agent displaces the ligand from binding to GPR124 or the fragment thereof. Preferably, a test agent is selected which displaces such ligand. The method may advantageously be conducted in a cell-free system.

For simplicity, test agents may initially be evaluated simply for binding to GPR124 or a fragment thereof, such as in a direct binding assay or a displacement assay as described above. Test agents which bind to GPR124 or displace the ligand from binding to GPR124 or the fragment thereof may additionally be evaluated in a mammalian cell, tissue or animal fibrosis model.

Thus, in a further aspect, the present invention is directed to a method of identifying a therapeutic agent for inhibiting transition of cells to myofibroblasts and/or reducing fibrosis, comprising:

contacting a test agent with G-protein coupled receptor (GPR124) or a fragment thereof;

detecting binding of said test agent to GPR124 or the fragment thereof;

identifying a test agent which binds to GPR124 or the fragment thereof as a candidate therapeutic agent;

testing the candidate therapeutic agent in a mammalian cell, tissue or animal fibrosis model to determine an expression level of one or more myofibroblast markers, extent of production of one or more ECM components, or extent of extracellular deposition of one or more ECM components in the cell, or in the tissue, or in an organ of said animal fibrosis model; and

selecting a candidate therapeutic agent that varies the expression level of one or more myofibroblast markers as compared to a reference expression level and/or that varies the extent of production and/or extracellular deposition of said ECM components as compared to a reference level of production and/or extracellular deposition for inhibiting transition of cells to myofibroblasts and/or reducing fibrosis.

In still a further aspect, the present invention is directed to a method of identifying a therapeutic agent for treatment, prevention or amelioration of a disorder or disease associated with or caused by excessive deposition of fibrous tissue comprising:

contacting a test agent with G-protein coupled receptor (GPR124) or a fragment thereof;

detecting binding of said test agent to GPR124 or the fragment thereof;

identifying a test agent which binds to GPR124 or the fragment thereof as a candidate therapeutic agent;

testing the candidate therapeutic agent in a mammalian cell, tissue or animal fibrosis model to determine an expression level of one or more myofibroblast markers, extent of production of one or more extracellular matrix (ECM) components, or extent of extracellular deposition of one or more ECM components in the cell, or in the tissue, or in an organ of said animal fibrosis model; and

selecting a candidate therapeutic agent that varies the expression level of one or more myofibroblast markers as compared to a reference expression level and/or that varies the extent of production and/or extracellular deposition of said ECM components as compared to a reference level of production and/or extracellular deposition for treating, preventing or ameliorating said disorder or disease.

Selected candidate therapeutic agents may serve as therapeutic agents according to the present invention.

Therapeutic agents according to the present invention may in particular be GPR124 antagonists.

Thus, the present invention provides methods for identifying therapeutic GPR124 antagonists. Several exemplary methods for identifying such antagonists are described herein, including cell-based and in vitro techniques. A general method of identifying GPR124 antagonists involves evaluating the effects of antagonist candidates on the level and/or activity of GPR124 under controlled conditions, such as in a binding assay conducted in a cell-free system, or in a cell-based assay.

Briefly, mammalian cells, tissues or a suitable test animal is treated with a predetermined dose of a GPR124 antagonist candidate. Control cells, control tissue or a control animal is treated with a control solution, preferably a non-irritating buffer solution or other carrier. The mammalian cells selected are optimally capable of transitioning to myofibroblasts and/or express GPR124. Exemplary cells include pericytes, fibroblasts, fibrocytes, endothelial cells, epithelial cells or mesenchymal stem cells. The cells are contacted with a candidate therapeutic agent, which is evaluated against control for effect of inhibition on level and/or activity of GPR124.

The level or activity of GPR124 may be determined directly or indirectly. Indirect determination may be obtained by comparing the expression level of one or more myofibroblast markers in cells induced to differentiate into myofibroblasts and contacted with the test agent with the expression level in control cells, as well as by comparing the extent of production or deposition of one or more extracellular matrix (ECM) component in media, the cell, tissue or organ.

For simplicity, candidate GPR124 inhibitors may initially be screened simply for binding to GPR124. In that event, it is initially expedient to utilize labeled candidate GPR124 inhibitors and/or labeled GPR124 and/or to conduct such initial investigation in a cell-free system. Either the candidate GPR124 inhibitor, or a soluble GPR124 fragment is desirably immobilized on a solid support. If a GPR124 fragment is utilized, the GPR124 fragment preferentially contains one or more GPR124 extracellular domains. Suitable extracellular domains include the leucine-rich repeat domain, leucine-rich repeat C-terminal domain, Ig domain and hormone receptor domain.

If warranted, GPR124 inhibitors identified in a cell-free system as binding GPR124 may thereafter be evaluated for reducing the level and/or activity of GPR124, or for reduced levels of myofibroblast markers or ECM production or deposition.

Identified effective candidates are suitable GPR124 antagonists that may be utilized for reducing pathological fibrosis or inhibiting transition of cells to myofibroblasts, as well as treating, preventing or ameliorating disorders and disease associated with or caused by excessive deposition of fibrous tissue.

The proteins of this invention, including fragments thereof, also may be used to raise monoclonal or polyclonal antibodies capable of binding specifically to an epitope of GPR124. These antibodies may be used, for example, in GPR124 antagonists purification protocols.

When the GPR124 antagonist candidate is delivered in a carrier, the control solution is ideally the carrier absent the GPR124 antagonist candidate. Multiple doses of the GPR124 antagonist candidate may be applied to the test animal, preferably following a predetermined schedule of dosing. The dosing schedule may be over a period of days, more preferably over a period of weeks.

A GPR124 antagonist candidate suitable for use as a GPR124 antagonist is identified by noting significant reduction in GPR124 activity and/or expression, and/or a significant reduction in myofibroblast markers or ECM component when compared to control. Ideally reduction of these indicators should be at least 10%, preferably 20%, further preferably 30% to 40% and most preferably 60% or more than is present in the control.

In an exemplary embodiment, localized injection in situ of a GPR124 antagonist candidate, for example a monoclonal antibody described herein, may be made into a test animal, with a control animal receiving an equal volume of control solution without the GPR124 antagonist candidate. Identical dosing should be done on a weekly basis for four weeks. Suitable dosage will depend on the nature of the particular GPR124 antagonist candidate being tested. By way of example, in dosing it should be noted that systemic injection, either intravenously, subcutaneously or intramuscularly, may also be used. For systemic injection of a GPR124 antagonist candidate, dosage should be in the range of from 0.01-200 mg/kg. Since it is typically conventional to utilize the lowest dosage necessary to achieve the desired clinical result, dosage may be preferably evaluated further from 0.01-100 mg/kg, more preferably from 0.01-50 mg/kg, advantageously from 0.01-25 mg/kg, more advantageously from 0.01-15 mg/kg, desirably from 0.01-10 mg/kg, more desirably from 0.01-1 mg/kg. Dosing performed by nebulized inhalation, eye drops, or oral ingestion should be at an amount sufficient to produce blood levels of the GPR124 antagonist candidate similar to those reached using systemic injection. The amount of GPR124 antagonist candidate that must be delivered by nebulized inhalation, eye drops, or oral ingestion to attain these levels is dependent upon the nature of the inhibitor used and can be determined by routine experimentation. It is expected that, for systemic injection of the monoclonal antibody GPR124 antagonist candidates described herein, therapeutic levels of the antibody may be detected in the blood one week after delivery of a 15 mg/kg dose.

GPR124 antagonists may also be identified using a process known as computer, or molecular modeling, which allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modelling system described generally above consists of the CHARMm and QUANTA programs, Polygen Corporation, Waltham, Mass. CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen, et al., Acta Pharmaceutica Fennica, Vol. 97 (1988) 159-66; Ripka, New Scientist (Jun. 16, 1988) 54-7; McKinaly and Rossmann, Annu. Rev. Pharmacol. Toxiciol., Vol. 29 (1989) 111-22; Perry and Davies, OSAR: Ouantitative Structure-Activity Relationships in Drug Design, Alan R. Liss, Inc. (1989)189-93; Lewis and Dean, Proc. R. Soc. Lond., Vol. 236 (1989) 25-162; and, with respect to a model receptor for nucleic acid components, Askew, et al., J. Am. Chem. Soc., Vol. 111 (1989) 1082-90. Askew et al. constructed a new molecular shape which permitted both hydrogen bonding and aromatic stacking forces to act simultaneously. Askew et al. used Kemp's triacid (Kemp et al., J. Org. Chem., Vol. 46 (1981) 5140-43) in which a U-shaped (diaxial) relationship exists between any two carboxyl functions. Conversion of the triacid to the imide acid chloride gave an acylating agent that could be attached via amide or ester linkages to practically any available aromatic surface. The resulting structure featured an aromatic plane that could be roughly parallel to that of the atoms in the imide function; hydrogen bonding and stacking forces converged from perpendicular directions to provide a microenvironment complimentary to adenine derivatives.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of drugs specific to regions of RNA, once that region is identified.

### 4. SCREENING COMPOUND LIBRARIES

GPR124 antagonists may desirably be further modified to enhance their therapeutic usefulness. This is typically done by creating large libraries of compounds related to the GPR124 antagonist, or compounds synthesized randomly, based around a core structure. In order to efficiently screen large and/or diverse libraries of GPR124 antagonist candidates, a high throughput screening method is necessary to at least decrease the number of candidate compounds to be screened using the assays described herein. High throughput screening methods involve providing a combinatorial chemical or peptide library containing a large number of potential therapeutic compounds (potential modulator or ligand compounds). Such “combinatorial chemical libraries” or “candidate libraries” are then screened in one or more assays, as described herein, to identify those library members (particular chemical species or subclasses) that are able to reduce fibrosis or inhibit transition of cells to myofibroblasts, as well as to treat, prevent or ameliorate disorders and diseases associated with or caused by excessive deposition of fibrous tissue. The compounds thus identified can serve as conventional “lead compounds” or can themselves be used as potential or actual therapeutics.

Accordingly, the present invention provides methods for high throughput screening of GPR124 antagonists candidates. The initial steps of these methods allow for the efficient and rapid identification of combinatorial library members that have a high probability of being GPR124 antagonists. Any method that determines the ability of a member of the library, termed a binding candidate, to specifically bind to GPR124 is suitable for this initial high throughput screening. For example, competitive and non-competitive ELISA-type assays known to one of ordinary skill in the art may be utilized.

Binding candidates that are found to bind GPR124 with acceptable specificity, e.g., with a Kₐ for GPR124 of at least about 10⁵ M⁻¹, 10⁶ M⁻¹ or greater, preferably 10⁷ M⁻¹ or greater, more preferably 10⁸ M⁻¹ or greater, and most preferably 10⁹ M¹ or greater, are GPR124 antagonist candidates and are screened further, as described herein, to determine their ability to reduce fibrosis or inhibit transition of cells to myofibroblasts, as well as to treat, prevent or ameliorate disorders and diseases associated with or caused by excessive deposition of fibrous tissue.

### 5. EXAMPLES

An increase in GPR124 mRNA expression was identified in mouse kidney tissues collected after unilateral ureteral obstruction (Example 1) and during diabetic nephropathy in vivo (Example 2). Likewise, treatment of NRK49F cells with TGF-beta under conditions that mimic kidney fibrosis in vitro lead to increased GPR124 mRNA expression (Example 3). GPR124 overexpression in NRK49F cells appears to drive a transition of the cells to myofibroblasts in response to TGF-beta and to lower extent even in absence of exogenously added TGF-beta (Example 4). An upregulation of GPR124 protein in the interstitium of adult kidney in a fibrosis model (Unilateral Ureteral Obstruction at day 10) was identified when compared to a control kidney (sham) (Example 5).

### Example 1: GPR124 is upregulated in a fibrosis model of Unilateral Ureteral Obstruction (UUO)

C57Bl/6J mice from Charles River Laboratories were allowed to adapt to housing conditions for one week. Surgery was performed on mice at 8 weeks of age. At time of surgery all mice weighed between 22 and 25 grams. Mice were anesthetized with pentobarbital sodium (60 mg/kg body weight) before surgery, and body temperatures were controlled at 36.5 to 37.5° C. throughout all procedures.

For the UUO procedure the left kidney was exposed through a flank incision and the left ureter tied off at the level of the lower pole with two 3.0 silk ties. One double knot and two single knots were used to cinch the ureter with each of the silk sutures. The mice were then allowed to recover on a heated pad and placed back into animal facility. For the control procedure (“sham”) mice were operated in identical fashion, except the ureter was not tied off with sutures.

Total kidney RNA was prepared at 3 days, 5 days, and 10 days following surgery. RNAseq analysis was performed using HiSeq Run Type, Single indexed, 2×100 bp, 8nt index run. N=4 mice/group. Data depicted as mean±SEM, Statistical analysis: 1-way ANOVA with Bonferroni post-hoc. *** p<0.0001; ** p<0.001

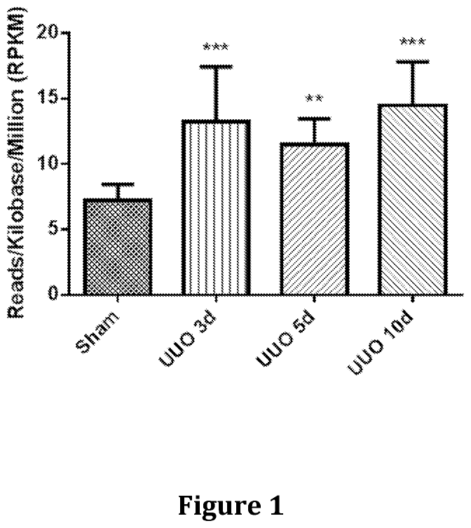

The results shown in FIG. 1 illustrate that GPR124 is upregulated in a mouse model of unilateral ureteral obstruction which is a generally acknowledged model of renal interstitial fibrosis.

### Example 2: GPR124 is Upregulated in a Model of Diabetic Nephropathy (Repeated Low Dose Streptozotocin (“STZ”) Induction)

C57Bl/6J mice from Charles River Laboratories were allowed to adapt to housing conditions for one week. First STZ was injected when mice were at 8 weeks of age. Before the first STZ injection, mice have been fasted for 4 hours. Freshly prepared STZ-Na citrate buffer solution (75 mg/kg per mouse) has been injected intra peritoneal (ip) daily on 5 consecutive days. Non-diabetic control animals were ip injected with Na citrate vehicle (0.05 M Na citrate, pH4.5), respectively.

Blood glucose measurements were used to confirm onset as well as persistence of diabetes over 8 weeks. If the blood glucose level of a mouse was >33 mmol/1 and weight was observed, mice received 0.125U insulin-glargine subcutaneous (sc).

Mice treated with STZ were sacrificed 8 weeks after diabetes onset as determined by blood glucose measurements. At this point in time, likewise non-diabetic control animals were sacrificed. All mice were sacrificed by first inducing anesthesia under isofluorane followed by systemic perfusion through the left ventricle with ice cold phosphate buffered saline. The left kidney was removed and dissected in an identical fashion in all mice. The papilla was removed and a sample of approximately 3 mm×2 mm was removed that contained the cortex and the medulla. The sample was flash frozen in liquid nitrogen and then stored at −80° C. Total tissue RNA was extracted using the standard protocol from Qiagen RNeasy Kit (kidney tissues) and Qiagen RNeasy Micro Kit (Cell lines). RNA was eluted in RNase-free water.

Work was conducted using RNase-free solutions and material at 4° C. on a 20-μL reaction volume for 1 ng-5 μg of total. Following components were added to a nuclease-free microcentrifuge tube: 1 μL of 250 ng/μL random primers, 1 μL dNTP Mix (10 mM each), 1 ng to 5 μg total RNA, x μL sterile, distilled water to 12 μL.

The mixture was heated to 65° C. for 5 min and rapidly transferred to 4° C. followed by brief centrifugation to spin down the solution and addition of 74, of the following mix: 4 μL, 5× First-Strand Buffer, 2 μl, 0.1 M DTT, 1 μL, RNaseOUT™ (40 units/μL).

Subsequently, samples were incubated at 25° C. for 2 min. 1 μL (200 units) of SuperScript™ II RT was added and samples were mixed by pipetting gently up and down. Using random primers, tubes were incubated at 25° C. for 10 min followed by incubate at 42° C. for 50 min. The reaction was stopped by heating to 70° C. for 15 min. Final cDNA samples were diluted with sterile, distilled water if required.

After the reverse transcription into cDNA the Real Time PCR was performed using Taqman® probe and the Taqman 7500 system. A PCR-Mix for a 12.5 μL-reaction was prepared as follows: 1.25 μL forward Primer (3 μM), 1.25 μL reverse Primer (3 μM), 1.25 μL Probe (2 μM), 6.25 μL TaqMan Universal Master Mix. Samples were transferred as duplicates into 96-well-plate, 2.5 μL of the cDNA sample (5 ng/μL) were added, and the plate was covered with optical adhesive tape. Plate was mixed and briefly centrifuged to collect the sample at the bottom of the plate.

### Thermal Cycler Protocol:

| | | |
|---|---|---|
| stage 1: | 2 min | 50° C. |
| stage 2: | 20 sec | 95° C. |
| stage 3: | 3 sec | 95° C. |
| 30 min | 59° C. | |

“stage 3” has been repeated 40 times

### Quantification and Normalization:

ΔC_{T}=C_{T} (target)−C_{T} (normalizer/calibrator/reference)

One reference sample was included as baseline. ΔΔC_{T} is the difference between each sample's ΔCT and the baseline's ΔCT. Comparative expression: 2−ΔΔCt is the fold expression relative to the reference.

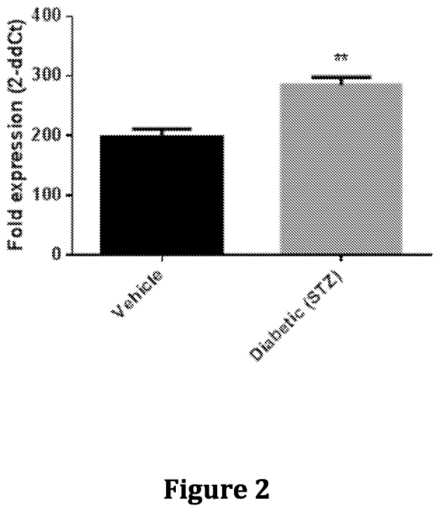

As evidenced in FIG. 2, the results show that GPR124 is upregulated in kidney tissue in a mouse model of diabetic nephropathy (repeated low-dose STZ). GPR124 mRNA expression in vehicle treated (non-diabetic) and STZ-treated (diabetic) mice has been quantified and normalized to 18s mRNA level by TaqMan technology. N=3 mice/group. Data depicted as mean±SEM, Statistical analysis: 1-way ANOVA with Bonferroni post-hoc. ** p<0.001

### Example 3: TGF-Beta Treatment Leads to Increased GPR124 mRNA Expression

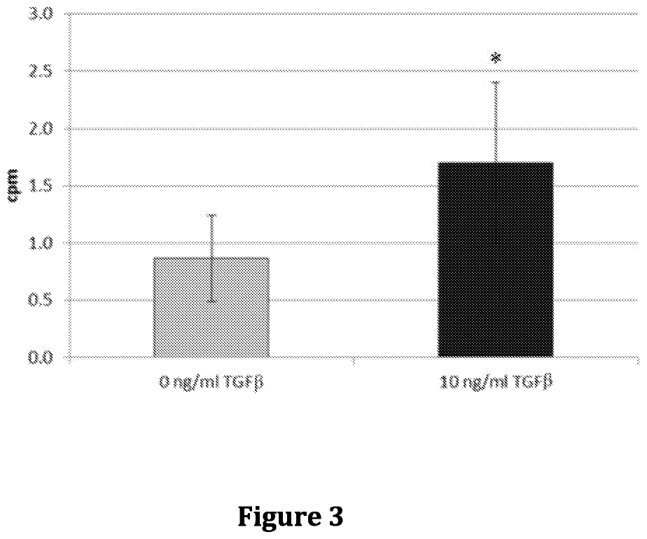

For cell expansion NRK49F cells (ATCC) were grown in Basal Media Eagle (Gibco, Billings, Mont.) with 5% fetal bovine serum supplemented with penicillin and streptomycin and 2 mmol/L glutamine. In order to monitor TGF-beta driven pericyte-to-myofibroblast transition, cells were grown on 6 well plates, serum starved by incubating in 0.5% fetal bovine serum for 12 hours, and then treated with TGF-beta at 2-10 ng/ml (category no. 100-21; PeproTech) for 12 to 48 hours for RT-PCR experiments. RNAseq analysis was performed using Illumina HiSeq 2000 system in 100 bp single read mode. As shown in FIG. 3, TGF-beta treatment leads to increased GPR124 mRNA expression in NRK49F kidney pericytes. N=3 per condition. Data depicted as mean±SEM, Statistical analysis: 1-way ANOVA with Bonferroni post-hoc. *p<0.01.

### Example 4: GPR124 Overexpression Leads to Myofibroblast Transition

In a further experiment, NRK49F cells were either transduced with GFP control virus or FLAG-tagged mouse GPR124 using the pLenti system followed by 4 weeks selection with 150 μg/ml G418. In order to monitor TGF-beta driven pericyte-to-myofibroblast transition, cells were grown on 6 well plates, serum starved by incubating in 0.5% fetal bovine serum for 12 hours, and then treated with TGF-beta at 2-10 ng/ml (category no. 100-21; PeproTech) for 12 to 48 hours for RT-PCR experiments.

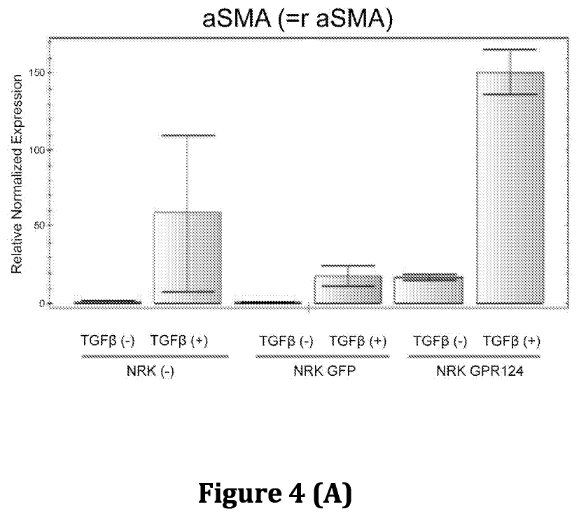

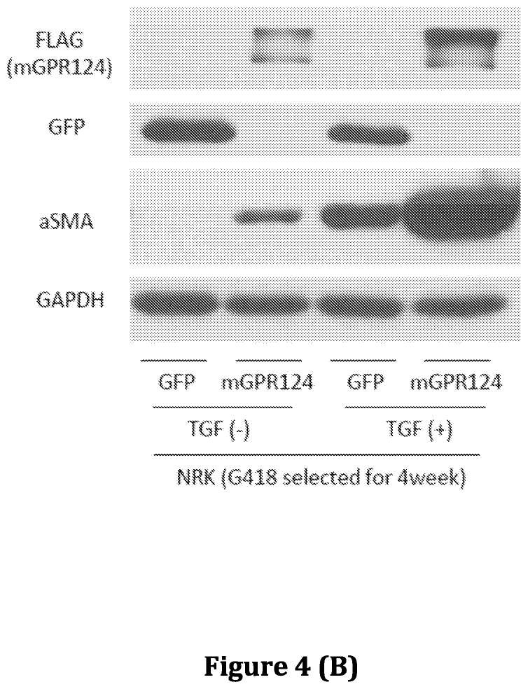

FIG. 4(A) illustrates quantifying rat alpha-SMA mRNA expression by qRT-PCR 24 h after treatment with TGF-beta (10 ng/ml) or vehicle control in NRK49F cells (“NRK(−)”), NRK49F cells transduced with GFP control (“NRK-GFP”) and NRK49F cells transduced with FLAG-tagged mouse GPR124 (“NRK-GPR124”). To measure protein expression, cell lysates were prepared in radioimmuno-precipitation assay buffer with protease inhibitors and phosphatase inhibitors, the total protein quantified by Bradford Assay and 20 μg separated by 10% polyacrylamide gel electrophoresis. Proteins were transferred to polyvinylidene difluoride membrane, blocked in 5% milk in phosphate buffered saline and, probed overnight at 4° C. with mouse anti-aSMA (Sigma-Aldrich, Cat. #A2547, 1:2000), or anti-FLAG rabbit anti-fibronectin (Abcam, Cambridge, Mass., Cat #ab23750, 1:6000), probed with anti-rabbit- or mouse-horseradish peroxidase (Dako, Carpinteria, Calif., 1:5000) for 1 hour at room temperature, and the antigen antibody complex was visualized using the ECL detection system (PerkinElmer, Waltham, Mass.).

FIG. 4(B) depicts expression of FLAG-tagged mGPR124 and GFP was monitored by Western Blot analysis using FLAG-tag- and GFP-specific antibodies, respectively. GFP control cells and FLAG-tagged mouse GPR124 expressing cells were either treated with 10 ng/ml TGF-beta or vehicle control for 24 h. Overexpression of FLAG-tagged mouse GPR124 leads to significant expression (Western Blot analysis) of the myofibroblast marker alpha-SMA, even in absence of TGF-beta. Both GFP-expressing and FLAG-tagged mouse GPR124 cells treated with 10 ng/ml TGF-beta for 24 h exhibit expression of alpha-SMA. Again, expression of FLAG-tagged mouse GPR124 leads to significantly higher expression of alpha-SMA when compared with GFP expressing cells. Analysis of GAPDH expression served as loading control.

When overexpressed in NRK49F cells, GPR124 appears to drive a transition of the cells to myofibroblasts in response to TGF-beta and to lower extent even in absence of exogenously added TGF-beta. Thus, pharmacological inhibition of the GPR124 receptor has the potential to reduce transition of cells to myofibroblasts and to reduce fibrosis as well as disorders and diseases associated therewith.

### Example 5: GPR124 Protein is Upregulated in Interstitium of Fibrotic Kidney as Assessed by Polyclonal Anti-Mouse GPR124 Immunohistochemistry

### Animals

All mouse experiments were performed according to the animal experimental guidelines issued by the Animal Care and Use Committee at Harvard University. C57Bl/6 mice were purchased from Jackson Laboratories (Bar Harbor, Me.). Unilateral ureteral obstruction (UUO) surgery was performed as previously described (Fabian et al., Am. J. Pathol. 180 (2012) 1441-1453). Briefly, after flank incision the left ureter was tied off at the level of the lower pole with two 4.0 silk ties. Mice were sacrificed at day 10 after surgery.

### Tissue Preparation and Histology

Mice were anesthetized with isofluorane (Baxter) and subsequently perfused via the left ventricle with 4° C. PBS for 1 minute. For histological analyses tissue sections were fixed in 10% formaldehyde for 1 hour, paraffin embedded and cut with a rotating microtome at 3 nm thickness and stained according to routine histology protocols. Immunohistochemistry was performed using a polyclonal antiserum raised against a C-terminal, cytoplasmic tail peptide of murine GPR124 (RDNLKGSGSALERESKRR) coupled to Keyhole limpet hemocyanin (KLH) and injected into rabbits according to standard protocols. Affinity purified antiserum was used at a 1:2000 dilution. A biotinylated secondary antibody was used (1:200, Jackson Immuno). Antigen retrieval was achieved by pressure cooker treatment and antigen unmasking solution (Vector). Staining was achieved using Avidin/Biotin Blocking kit, the ABC kit, the DAB kit and the DAB enhancing solution (all Vector laboratories) according to manufacturer instructions.

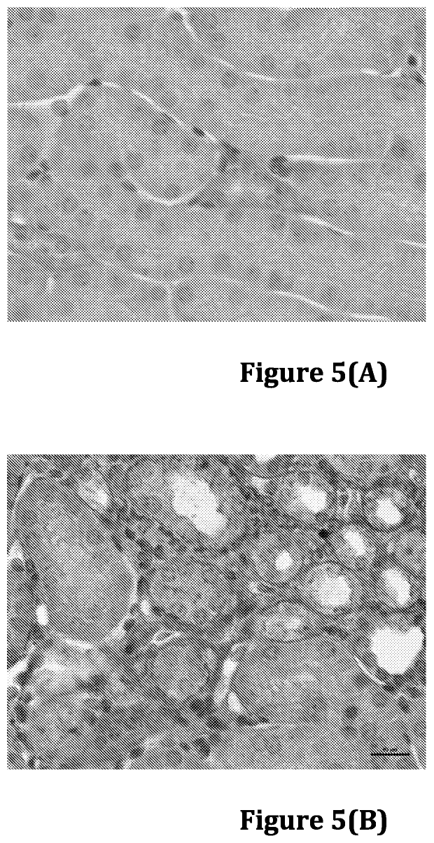

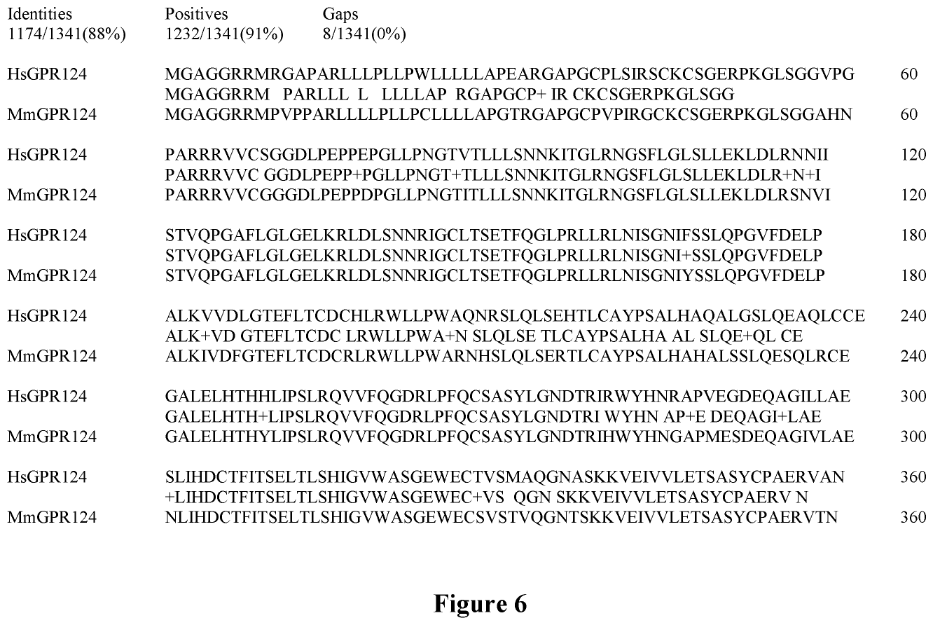

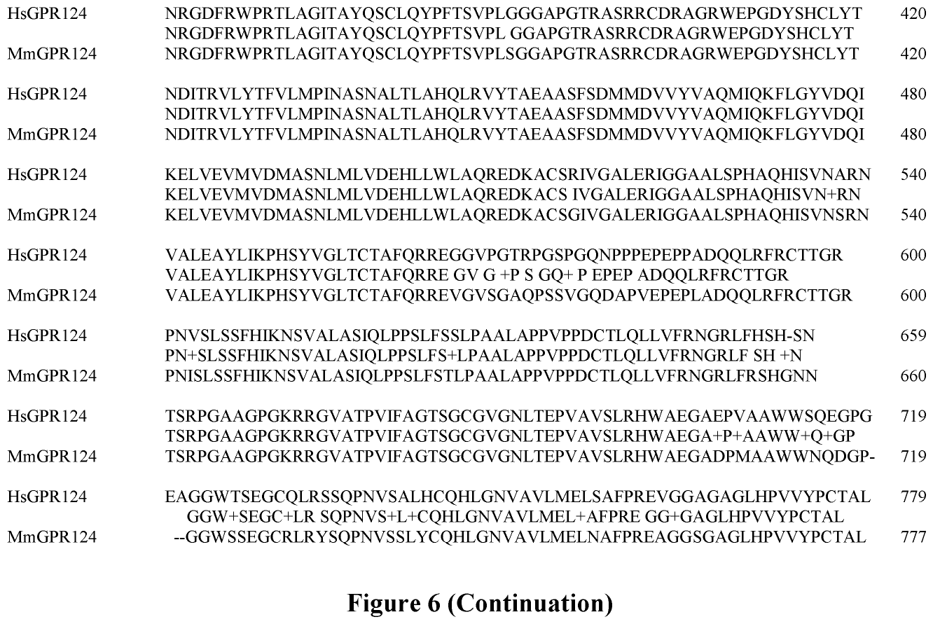

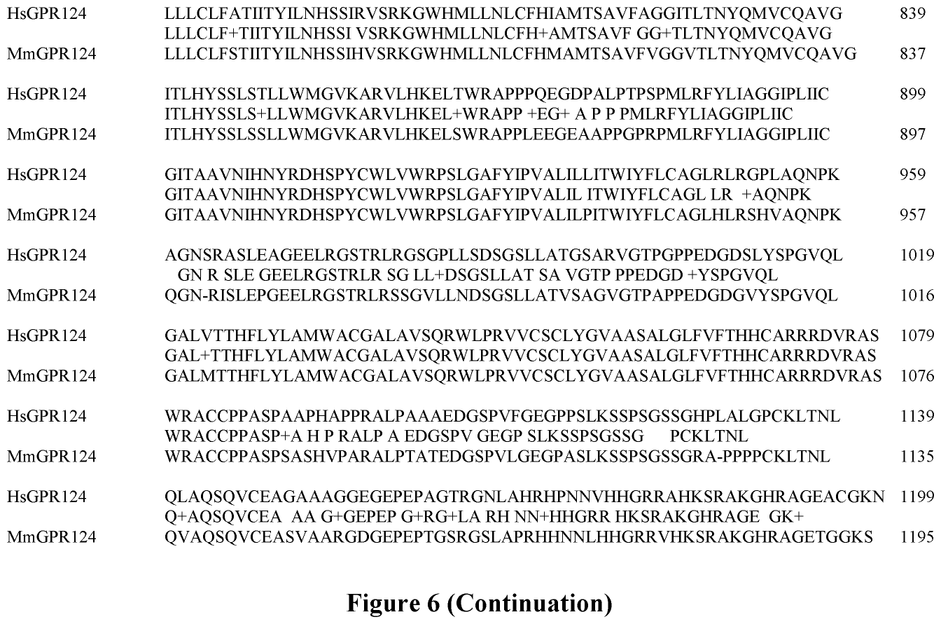

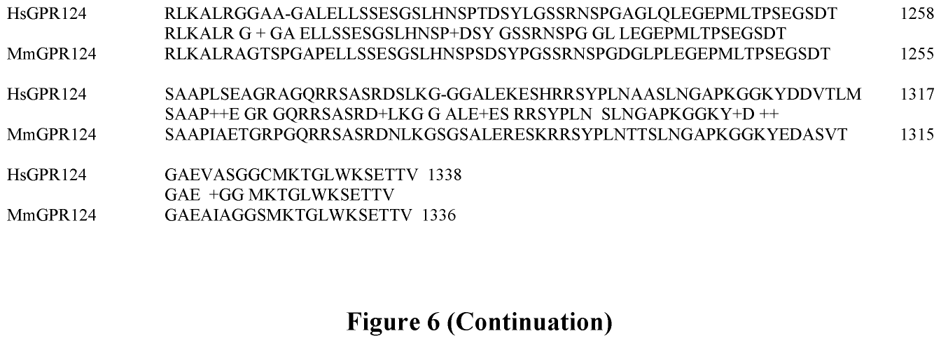

As shown in FIG. 5(A) in healthy adult kidney (sham), faint GPR124 staining is observed in the interstitial space, consistent with a pericyte localization. At day 10, there is intense staining limited to the interstitial space, consistent with mGRP124 expression in myofibroblasts (FIG. 5(B)).

### 6. THERAPEUTIC APPLICATIONS

Accordingly, in view of the foregoing, it is understood that pathological fibrosis can be reduced, and disease and disorders associated with or caused by excessive deposition of fibrous tissue in a mammalian subject, can be treated, prevented or ameliorated by administering a suitable agent that modulates the level and/or activity of GPR124. In one embodiment, the agent may reduce the activity of GPR124 in cells undergoing transition to myofibroblasts, wherein the cells may be pericytes, fibroblasts, fibrocytes, endothelial cells, epithelial cells or mesenchymal stem cells. In another embodiment, the pathological fibrosis, or disease or disorder may impair the architecture and/or function of organs, such as kidney, liver, lung, heart or skin. Further organs which may be impaired include pancreas, vascular vessels, bone marrow and the like. Such disorders, diseases and pathological fibroses may for example include diabetic nephropathy, focal segmental glomerulosclerosis, minimal change disease, chronic kidney disease, end stage renal disease, pre-stage liver cirrhosis, liver cirrhosis, pulmonary fibrosis, cardiac fibrosis or scleroderma. The pulmonary fibrosis may be idiopathic pulmonary fibrosis or pulmonary cystic fibrosis. The cardiac fibrosis may be myocardial fibrosis such as endomyocardial fibrosis.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

1-59. (canceled)

60. A method for reducing pathological fibrosis in a mammalian subject in need thereof, comprising down modulating the level and/or activity of G-protein coupled receptor 124 (GPR124) in said subject by administering a therapeutic agent to a subject in need thereof, wherein
said therapeutic agent is an antibody, a peptide or a nonpeptide molecule, and
said G-protein coupled receptor 124 comprising the sequence of either SEQ ID NOS: 3 or 4, or a polypeptide homologous thereto.

61. The method according to claim 60, which reduces the level and/or activity of GPR124 in cells undergoing transition to myofibroblasts.

62. The method according to claim 61, wherein the cells are selected from the group consisting of pericytes, fibroblasts, fibrocytes, endothelial cells, epithelial cells and mesenchymal stem cells.

63. The method according to claim 60, wherein said pathological fibrosis impairs architecture and/or function of an organ selected from the group consisting of kidney, liver, lung, heart, pancreas, vascular vessel, bone marrow and skin.

64. The method according to claim 63, wherein said pathological fibrosis is a disorder or disease selected from the group consisting of diabetic nephropathy, focal segmental glomerulosclerosis (FSGS), minimal change disease (MCD), chronic kidney disease (CKD) and end stage renal disease (ESRD), or said pathological fibrosis is associated with said disorder or disease.

65. The method according to claim 63, wherein said pathological fibrosis is liver cirrhosis or a pre-stage thereof, or said pathological fibrosis is associated with liver cirrhosis or pre-stage thereof.

66. The method according to claim 63, wherein said pathological fibrosis is idiopathic pulmonary fibrosis or pulmonary cystic fibrosis, or said pathological fibrosis is associated with idiopathic pulmonary fibrosis or pulmonary cystic fibrosis.

67. The method according to claim 63, wherein said pathological fibrosis is myocardial fibrosis.

68. The method according to claim 63, wherein said pathological fibrosis is scleroderma, or said pathological fibrosis is associated with scleroderma.

69. A method for treating, preventing or ameliorating a disorder or disease associated with or caused by excessive deposition of fibrous tissue in a mammalian subject in need thereof, comprising down modulating the level and/or activity of G-protein coupled receptor 124 (GPR124) in said subject by administering a therapeutic agent to a subject in need thereof, wherein
said therapeutic agent is an antibody, a peptide or a nonpeptide molecule, and
said G-protein coupled receptor 124 comprising the sequence of either SEQ ID NOS: 3 or 4, or a polypeptide homologous thereto.

70. The method according to claim 69, which reduces the level and/or activity of GPR124 in cells undergoing transition to myofibroblasts.

71. The method according to claim 70, wherein the cells are selected from the group consisting of pericytes, fibroblasts, fibrocytes, endothelial cells, epithelial cells and mesenchymal stem cells.

72. The method according to claim 69, wherein said disorder or disease impairs architecture and/or function of an organ selected from the group consisting of kidney, liver, lung, heart, pancreas, vascular vessel, bone marrow and skin.

73. The method according to claim 72, wherein said disorder or disease is selected from the group consisting of diabetic nephropathy, focal segmental glomerulosclerosis (FSGS), minimal change disease (MCD), chronic kidney disease (CKD) and end stage renal disease (ESRD).

74. The method according to claim 72, wherein said disorder or disease is liver cirrhosis or a pre-stage thereof.

75. The method according to claim 72, wherein said disorder or disease is idiopathic pulmonary fibrosis or pulmonary cystic fibrosis.

76. The method according to claim 72, wherein said disorder or disease is myocardial fibrosis.

77. The method according to claim 72, wherein said disorder or disease is scleroderma.

78. A method for reducing pathological fibrosis of a kidney in a mammalian subject in need thereof, comprising reducing the level and/or activity of G-protein coupled receptor 124 (GPR124) in said subject by administering a therapeutic agent to a subject in need thereof, wherein
said therapeutic agent is an antibody, a peptide or a nonpeptide molecule, and
said G-protein coupled receptor 124 comprising the sequence of either SEQ ID NOS: 3 or 4, or a polypeptide homologous thereto.

79. The method of claim 78, comprising reducing the level and/or activity of G-protein coupled receptor 124 (GPR124) in said subject, wherein said therapeutic agent is a GPR124 antagonist.


SEQUENCE LISTING (4 sequences)

SEQ ID NO: 1 - DNA, 6050 bp, Human
  Adhesion G protein-coupled receptor A2 (ADGRA2) NCBI Reference Sequence NM_032777.9
atccatggcacggagcggcggcggcggcggcagcaggagcccggcgcgatccgctaggtcccagcccagcgcccagcgagcaggcgacgcggaggggccgggcctccagtgtcccgagggccgggcgctgagactccggccgcgcagctgggagctgcccgcgctgcgctgacagccgcgccgacgtcctccccgccggggcgctcgcaggacatgcccccggggcgcggcggcggggaccccggggctcgcctccgcccagggcccccctccacgccctcgggagccccgggcccccgctgagcactcctcccgcacgcctgggtccctccggccggcgcgcagcccggccccagcgctgtgggtccccgcggggcgatgggttgatgggcgccgggggacgcaggatgcggggggcgcccgcgcgcctgctgctgccgctgctgccgtggctcctgctgctcctggcgcccgaggctcggggcgcgcccggctgcccgctatccatccgcagctgcaagtgctcgggggagcggcccaaggggctgagcggcggcgtccctggcccggctcggcggagggtggtgtgcagcggcggggacctcccggagcctcccgagcccggccttctgcctaacggcaccgttaccctgctcttgagcaataacaagatcacggggctccgcaatggctccttcctgggactgtcactgctggagaagctggacctgaggaacaacatcatcagcacagtgcagccgggcgccttcctgggcctgggggagctgaagcgtttagatctctccaacaaccggattggctgtctcacctccgagaccttccagggcctccccaggcttctccgactaaacatatctggaaacatcttctccagtctgcaacctggggtctttgatgagctgccagcccttaaggttgtggacttgggcaccgagttcctgacctgtgactgccacctgcgctggctgctgccctgggcccagaatcgctccctgcagctgtcggaacacacgctctgtgcttaccccagtgccctgcatgctcaggccctgggcagcctccaggaggcccagctctgctgcgagggggccctggagctgcacacacaccacctcatcccgtccctacgccaagtggtgttccagggggatcggctgcccttccagtgctctgccagctacctgggcaacgacacccgcatccgctggtaccacaaccgagcccctgtggagggtgatgagcaggcgggcatcctcctggccgagagcctcatccacgactgcaccttcatcaccagtgagctgacgctgtctcacatcggcgtgtgggcctcaggcgagtgggagtgcaccgtgtccatggcccaaggcaacgccagcaagaaggtggagatcgtggtgctggagacctctgcctcctactgccccgccgagcgtgttgccaacaaccgcggggacttcaggtggccccgaactctggctggcatcacagcctaccagtcctgcctgcagtatcccttcacctcagtgcccctgggcgggggtgccccgggcacccgagcctcccgccggtgtgaccgtgccggccgctgggagccaggggactactcccactgtctctacaccaacgacatcaccagggtgctgtacaccttcgtgctgatgcccatcaatgcctccaatgcgctgaccctggctcaccagctgcgcgtgtacacagccgaggccgctagcttttcagacatgatggatgtagtctatgtggctcagatgatccagaaatttttgggttatgtcgaccagatcaaagagctggtagaggtgatggtggacatggccagcaacctgatgctggtggacgagcacctgctgtggctggcccagcgcgaggacaaggcctgcagccgcatcgtgggtgccctggagcgcattgggggggccgccctcagcccccatgcccagcacatctcagtgaatgcgaggaacgtggcattggaggcctacctcatcaagccgcacagctacgtgggcctgacctgcacagccttccagaggagggagggaggggtgccgggcacacggccaggaagccctggccagaaccccccacctgagcccgagcccccagctgaccagcagctccgcttccgctgcaccaccgggaggcccaatgtttctctgtcgtccttccacatcaagaacagcgtggccctggcctccatccagctgcccccgagtctattctcatcccttccggctgccctggctcccccggtgcccccagactgcaccctgcaactgctcgtcttccgaaatggccgcctcttccacagccacagcaacacctcccgccctggagctgctgggcctggcaagaggcgtggcgtggccacccccgtcatcttcgcaggaaccagtggctgtggcgtgggaaacctgacagagccagtggccgtttcgctgcggcactgggctgagggagccgaacctgtggccgcttggtggagccaggaggggcccggggaggctgggggctggacctcggagggctgccagctccgctccagccagcccaatgtcagcgccctgcactgccagcacttgggcaatgtggccgtgctcatggagctgagcgcctttcccagggaggtggggggcgccggggcagggctgcaccccgtggtatacccctgcacggccttgctgctgctctgcctcttcgccaccatcatcacctacatcctcaaccacagctccatccgtgtgtcccggaaaggctggcacatgctgctgaacttgtgcttccacatagccatgacctctgctgtctttgcggggggcatcacactcaccaactaccagatggtctgccaggcggtgggcatcaccctgcactactcctccctatccacgctgctctggatgggcgtgaaggcgcgagtgctccataaggagctcacctggagggcaccccctccgcaagaaggggaccccgctctgcctactcccagtcctatgctccggttctatttgatcgctggagggattccactcattatctgtggcatcacagctgcagtcaacatccacaactaccgggaccacagcccctactgctggctggtgtggcgtccaagccttggcgccttctacatccctgtggctttgattctgctcatcacctggatctatttcctgtgcgccgggctacgcttacggggtcctctggcacagaaccccaaggcgggcaacagcagggcctccctggaggcaggggaggagctgaggggttccaccaggctcaggggcagcggccccctcctgagtgactcaggttcccttcttgctactgggagcgcgcgagtggggacgcccgggcccccggaggatggtgacagcctctattctccgggagtccagctaggggcgctggtgaccacgcacttcctgtacttggccatgtgggcctgcggggctctggcagtgtcccagcgctggctgccccgggtggtgtgcagctgcttgtacggggtggcagcctccgccctgggcctcttcgtcttcactcaccactgtgccaggcggagggacgtgagagcctcgtggcgcgcctgctgcccccctgcctctcccgcggccccccatgccccgccccgggccctgcccgccgccgcagaggacggttccccggtgttcggggagggccccccctccctcaagtcctccccaagcggcagcagcggccatccgctggctctgggcccctgcaagctcaccaacctgcagctggcccagagtcaggtgtgcgaggcgggggcggcggccggcggggaaggagagccggagccggcgggcacccggggaaacctcgcccaccgccaccccaacaacgtgcaccacgggcgtcgggcgcacaagagccgggccaagggacaccgcgcgggggaggcctgcggcaagaaccggctcaaggccctgcgcgggggcgcggcgggggcgctggagctgctgtccagcgagagcggcagtctgcacaacagccccaccgacagctacctgggcagcagccgcaacagcccgggcgccggcctgcagctggaaggcgagcccatgctcacgccgtccgagggcagcgacaccagcgccgcgccgctttctgaggcgggccgggcaggccagcgccgcagcgccagccgcgacagtctcaagggcggcggcgcgctggagaaggagagccatcgccgctcgtacccgctcaacgccgccagcctaaacggcgcccccaaggggggcaagtacgacgacgtcaccctgatgggcgcggaggtagccagcggcggctgcatgaagaccggactctggaagagcgaaactaccgtctaaggtggggcgggcgacgcggtagacgggctggccacgcggctcgttcccccgctcctcggggccctccaaggtgtctccgtagtcagcaggttggaggcagaggagccgatggctggaggaagcccacaggcggatgttccccacttgcctagagggcatccctctggggtagcgacagacaatcccagaaacacgcataatacatttccgtccagcccggggcagtctgactgtcggtgccctcccaggaacggggaaggcctccgtctgtgtgaaagggcacagcacatcccaggtgcaccctccccaagtactcccaccccgcctactgtccatgcggcctcactgggggccatcagcctcaccagcaaagcagagatgagagcgtgggaactgtgttctttcctccctgccctctactgatttcagcccagcccctgcctagatcctaggtcccttttcctcccgagtttggctggcacgagagctagcccagcacatgaagcaggtgatgttaagtcacaaggtgctgcttttcagatccactatgcaagaggggagggtggggccacgtgaaaggcagctctagacatcaaccagtcctgggggaggggagtgggaaccgggcacaactaggaacaatgccaccattcccacaggagtggtacttaaaccagacagcagggttcagaggtggcacaccgggacaaagctgaggccctgcacctcaacagctgactgccaggtgcctgtgggtgaactgaggggagtagagggagagggcaggtggaactggggcagaatctagtcatgccctaaagctagtcctgtaaacaatggtgccccagaaagctgcaggtggtgtttggagaagcagttacttttcagttacaagacccatctccctagtctcagccttacaacaccacgggactaaggaagagcacttccttgcctccgtaaggccagaggaagaaccatcccaatcatttgatctccagctccacagtagagagaaacctacaaaatgtcaaaccagcttcccgactcccaggagctcaagccaagcccagaggcagtggctggggtccctgcaggtcatgaggggcctatgcctttactccttttaaacaccagcacccgtcttttccccaacctaaaaccaaccaccagcatttcactacaggaccaaatggaaaccgagggaaccctgggtcttgggaagaacaacaggaaaccaaggtctgacctagggttccctcccagtcttcacatcactctggcctcatcaccaaggtgacagaggacacaggggagggggaaaacccacacacactccttggaatgggtcctgttatttatgcttgctgcacagacatattagaagaaaaaaaaaagctttgtattattcttccacatatgctggctgctgtttacacaccctgccaatgccttagcactggagagctttttgcaatatgctggggaaaggggagggagggaatgaaagtgccaaagaaaacatgtttttaagaactcgggttttatacaatagaatgttttctagcagatgcctcttgttttaatatattaaaattttgcaaagccctttgagctactgccttagtctaaaaaaaaaaaaaaaaa

SEQ ID NO: 2 - DNA, 4475 bp, Mus musculus
  Adhesion G protein-coupled receptor A2 (Adgra2) NCBI Reference Sequence NM_133911.1
ggcggcgctcccgcgggccgagcggcgagctgggacccggcggccctgaccccgcggctccgcggagggcgagcgcggccgcggacaaaggagcatgtcgtcgcgaagcgggaccgatcctcgggccgccgggaggctcgggccaccgccagggccgccccgccccgccgccgcccgctaggccggtgccgtctctcggccccgcaggcccggtcagcggcatggagccgccgccgccgctgctgctgctgccgctggcgctgctcgctctgctgtggggaggagagcgcggggccgcggcgctgcccgcgggctgcaagcacgacggtcgggcccggggtaccggccgagcggccgccgccgccgaggggaaggtggtgtgcagcagcctggagctcgcgcaggttctgcccccggacacgctgcccaaccgcacggtcaccctgattttaagcaacaacaagatctccgagctgaagaatggttcattttctggcttaagtctcctcgaaagactggacctccggaacaaccttattagtaggatagccccaggtgccttttggggactgtcttcactgaagagattggacctgacgaacaaccgaataggttgtctgaatgcagatgtatttcgaggactcaccaatctggttcggctaaacctttcagggaatctgtttacttcactgtctcaaggaacttttgattatcttggctcgttgcggtctttagaatttcagactgagtaccttctgtgtgactgtaacattctgtggatgcatcgctgggtaaaggagagaaacatcactgtgcgagacaccaggtgtgtttatcccaagtcactgcaggcccagcctgtcacgggggtgaaacaggagctcctcacttgcgatcctccccttgaactgccgtccttctacatgactccgtcgcaccgccaggttgtgtttgaaggagacagccttcccttccagtgcatggcttcctatatagatcaggacatgcaagtgctgtggtatcaggatgggcgcattgttgagaccgatgagtcccaagggatctttgtggagaaaagcatgattcacaattgctccttgatcgccagtgccctaaccatttctaatattcaagctggatctactggaaattggggctgtcatgttcagacgaaacgtgggaataacacaagaactgttgacattgtggtattagaaagctccgcccaatactgtccaccagagagggttgtgaacaacaaaggtgatttcagatggcccaggacactggcgggcatcacagcatatctccagtgtacccggaacacccacagcagtgggatctaccctggaagcgcacaggatgaaaggaaggcgtggcgccgatgcgacagaggtggcttttgggcagatgatgattattctaggtgccagtatgcaaatgacgtcactagagtcctgtatatgtttaatcagatgcccctcaaccttacaaatgcggtcgctacagctcggcagctgctggcttacacagtggaggccgccaacttctctgacaaaatggacgttatatttgtggctgaaatgatagaaaagtttggaagatttaccagagaggaaaaatcaaaagagcttggtgatgtaatggtcgatgtggcaagcaacatcatgttggctgatgagcgggtcctgtggctggcacagagggaagcaaaggcctgcagtcggattgtccagtgcctgcagcgcattgccacacatcgcctggccagtggggcccacgtgtactccacgtactcgcccaacattgctctggaggcttacgtcatcaaggctgctggcttcacaggaatgacctgctccgtgttccagaaggtggctgcctccgaccgtgcaggtctttctgactatgggcgaagggacccggatggaaacctggataagcagctgagcttcaaatgcaatgtctccagcaccttctcaagcctggccctgaagaacaccatcatggaggcctccattcagcttccttcctcccttttgtcaccaaaacacaagcgagaagcccgagcggcggatgacgccctctataagctccagctcattgccttccgcaacggaaagctttttccagccacaggaaattcaacaaagttggcagacgatggcaagcggcggacagtagtgacccctgtgatcctcacgaaaatagatggtgcaaccgtagatacccaccacatccctgttaatgtgacgctgcgccgaattgcccacggagcagatgcggttgcagcgcagtgggactttgatttgctgaacggacaaggaggctggaagtcagatgggtgctgtatactctactcggatgagaacatcaccaccattcagtgcggctccctgggcaactatgctgtgctaatggatctgactgggacagagttgtacaccccagcagccagtctcctgcaccctgtggtttacaccactgccatcactctcctcttgtgtctcttggctgttatcatcagttacatgtaccaccacagcttgatccgaatcagtctcaagagctggcacatgcttgtgaacctgtgctttcacattctcctgacctgcgtggtgtttgtgggaggaataacccagaccagaaatgccagcgtctgtcaagcagttgggatcattcttcattattctacccttgccacggtattgtgggttggagtcactgctagaaatatctataaacaagtcaccaagaaagccaagagatgccaggatccagatgagccacccgctcctccacgaccgatgctgaggttctacctgattggtggtgggatccccatcatagtgtgtggtatcaccgcggcagcaaacatcaagaactatggcagtcggcccagtgcaccgtattgctggatggcctgggaaccgtccttgggagccttctacggacctgccagcttcatcacttttgtaaactgtatgtattttctaagcatatttattcagttgaaaagacaccctgagcgcaaatatgagctcaaggagccgacagaagagcagcagagattggcagccaatgaaaatggtgaaatcaaccatcaggactccatgtctctgtctctcatctctacgtccacgttggagaacgagcacagttttcagtctcagcttctgggcgccagccttactttgcttttgtatgtcatcttgtggatgtttggggccatggctgtttctctgtattaccctctggacttggtttttagcttcttcttcggagccacttgtttaagcttcagtgctttcatgatggtgcaccactgcatcaacagggaggacgtgagacttgcgtggatcatgatgtgctgcccagggcggagctcgtactccgtgcaagtcaacgtccaacctcccaactcaagcgccactaatggagaggctccaaagtgcaccaatagcagcgcagagtcttcgtgcacgaacaaaagcgcatcgagcttcaaaaactcttcccagggctgcaagctgacaaacttgcaggctgctgcggcacagtaccacagcaatgccctacctgtgaatgccacgccgcagcttgataacagtctgaccgaacactcgatggacaacgatattaaaatgcatgtggctcctttagacgtgcagtttcgaacaaacgtgcacccaagccgccaccacaaaaaccgaagtaaaggacaccgggccagcaggctcacagtcctgcgagagtatgcctatgacgtcccaacaagtgtggaaggaagcgtgcagaatggcttacctaaaagccggccaggcagcaatgaaggacattcaaggagtaggagagcttatttagcctacagagagagacagtacaacccaccccaacaagacagcagtgatgcttgtagcacacttcccaaaagtagccgaaatgttgaaaagcctgtttcaactagtagtaagaaagatgcaccaaggaagccagctgcagccgaccttgaaagtcagcagaaatcttacggcctgaacttggctgttcagaatggaccagttaaaagcaatgggcaggaaggacccttgctagctaccgacgtcactggtaatgttaggactgggttatggaaacacgaaacaactgtgtagcattgcaggggcttccaagacaaggtgaaactgtggtactcacattcctttaagctatgaactcttagaaacaaactgtttacagccaccttggggatacaaagccgttctgagtattcttgtgagctttgggatttacttattttatattcccaaattgtcccccactccccccaagagttaaaaatgttttaaaacattgttttacttgtcaaagcaccaataagatattttggaagttgaaaatataatttcttagaatctgttatatctgcgtaacatctgagacttgtatttaataaactaaatagaagtttgtca

SEQ ID NO: 3 - protein, 1338 aa, Human
  G-protein coupled receptor 124 precursor including signal peptide Sequence ID refNP_116166.9
MGAGGRRMRGAPARLLLPLLPWLLLLLAPEARGAPGCPLSIRSCKCSGERPKGLSGGVPGPARRRVVCSGGDLPEPPEPGLLPNGTVTLLLSNNKITGLRNGSFLGLSLLEKLDLRNNIISTVQPGAFLGLGELKRLDLSNNRIGCLTSETFQGLPRLLRLNISGNIFSSLQPGVFDELPALKVVDLGTEFLTCDCHLRWLLPWAQNRSLQLSEHTLCAYPSALHAQALGSLQEAQLCCEGALELHTHHLIPSLRQVVFQGDRLPFQCSASYLGNDTRIRWYHNRAPVEGDEQAGILLAESLIHDCTFITSELTLSHIGVWASGEWECTVSMAQGNASKKVEIVVLETSASYCPAERVANNRGDFRWPRTLAGITAYQSCLQYPFTSVPLGGGAPGTRASRRCDRAGRWEPGDYSHCLYTNDITRVLYTFVLMPINASNALTLAHQLRVYTAEAASFSDMMDVVYVAQMIQKFLGYVDQIKELVEVMVDMASNLMLVDEHLLWLAQREDKACSRIVGALERIGGAALSPHAQHISVNARNVALEAYLIKPHSYVGLTCTAFQRREGGVPGTRPGSPGQNPPPEPEPPADQQLRFRCTTGRPNVSLSSFHIKNSVALASIQLPPSLFSSLPAALAPPVPPDCTLQLLVFRNGRLFHSHSNTSRPGAAGPGKRRGVATPVIFAGTSGCGVGNLTEPVAVSLRHWAEGAEPVAAWWSQEGPGEAGGWTSEGCQLRSSQPNVSALHCQHLGNVAVLMELSAFPREVGGAGAGLHPVVYPCTALLLLCLFATIITYILNHSSIRVSRKGWHMLLNLCFHIAMTSAVFAGGITLTNYQMVCQAVGITLHYSSLSTLLWMGVKARVLHKELTWRAPPPQEGDPALPTPSPMLRFYLIAGGIPLIICGITAAVNIHNYRDHSPYCWLVWRPSLGAFYIPVALILLITWIYFLCAGLRLRGPLAQNPKAGNSRASLEAGEELRGSTRLRGSGPLLSDSGSLLATGSARVGTPGPPEDGDSLYSPGVQLGALVTTHFLYLAMWACGALAVSQRWLPRVVCSCLYGVAASALGLFVFTHHCARRRDVRASWRACCPPASPAAPHAPPRALPAAAEDGSPVFGEGPPSLKSSPSGSSGHPLALGPCKLTNLQLAQSQVCEAGAAAGGEGEPEPAGTRGNLAHRHPNNVHHGRRAHKSRAKGHRAGEACGKNRLKALRGGAAGALELLSSESGSLHNSPTDSYLGSSRNSPGAGLQLEGEPMLTPSEGSDTSAAPLSEAGRAGQRRSASRDSLKGGGALEKESHRRSYPLNAASLNGAPKGGKYDDVTLMGAEVASGGCMKTGLWKSETTV

SEQ ID NO: 4 - protein, 1336 aa, Mus musculus
  G-protein coupled receptor 124 precursor including signal peptide Sequence ID refNP_473385.2
MGAGGRRMPVPPARLLLLPLLPCLLLLAPGTRGAPGCPVPIRGCKCSGERPKGLSGGAHNPARRRVVCGGGDLPEPPDPGLLPNGTITLLLSNNKITGLRNGSFLGLSLLEKLDLRSNVISTVQPGAFLGLGELKRLDLSNNRIGCLTSETFQGLPRLLRLNISGNIYSSLQPGVFDELPALKIVDFGTEFLTCDCRLRWLLPWARNHSLQLSERTLCAYPSALHAHALSSLQESQLRCEGALELHTHYLIPSLRQVVFQGDRLPFQCSASYLGNDTRIHWYHNGAPMESDEQAGIVLAENLIHDCTFITSELTLSHIGVWASGEWECSVSTVQGNTSKKVEIVVLETSASYCPAERVTNNRGDFRWPRTLAGITAYQSCLQYPFTSVPLSGGAPGTRASRRCDRAGRWEPGDYSHCLYTNDITRVLYTFVLMPINASNALTLAHQLRVYTAEAASFSDMMDVVYVAQMIQKFLGYVDQIKELVEVMVDMASNLMLVDEHLLWLAQREDKACSGIVGALERIGGAALSPHAQHISVNSRNVALEAYLIKPHSYVGLTCTAFQRREVGVSGAQPSSVGQDAPVEPEPLADQQLRFRCTTGRPNISLSSFHIKNSVALASIQLPPSLFSTLPAALAPPVPPDCTLQLLVFRNGRLFRSHGNNTSRPGAAGPGKRRGVATPVIFAGTSGCGVGNLTEPVAVSLRHWAEGADPMAAWWNQDGPGGWSSEGCRLRYSQPNVSSLYCQHLGNVAVLMELNAFPREAGGSGAGLHPVVYPCTALLLLCLFSTIITYILNHSSIHVSRKGWHMLLNLCFHMAMTSAVFVGGVTLTNYQMVCQAVGITLHYSSLSSLLWMGVKARVLHKELSWRAPPLEEGEAAPPGPRPMLRFYLIAGGIPLIICGITAAVNIHNYRDHSPYCWLVWRPSLGAFYIPVALILPITWIYFLCAGLHLRSHVAQNPKQGNRISLEPGEELRGSTRLRSSGVLLNDSGSLLATVSAGVGTPAPPEDGDGVYSPGVQLGALMTTHFLYLAMWACGALAVSQRWLPRVVCSCLYGVAASALGLFVFTHHCARRRDVRASWRACCPPASPSASHVPARALPTATEDGSPVLGEGPASLKSSPSGSSGRAPPPPCKLTNLQVAQSQVCEASVAARGDGEPEPTGSRGSLAPRHHNNLHHGRRVHKSRAKGHRAGETGGKSRLKALRAGTSPGAPELLSSESGSLHNSPSDSYPGSSRNSPGDGLPLEGEPMLTPSEGSDTSAAPIAETGRPGQRRSASRDNLKGSGSALERESKRRSYPLNTTSLNGAPKGGKYEDASVTGAEAIAGGSMKTGLWKSETTV